(19) United States
(12) Patent Application Publication
(10) Pub. No.: US 2016/0228036 A1
(43) Pub. Date: Aug. 11, 2016

(54) WIRELESS CAPNOGRAPHY

(71) Applicant: ORIDION MEDICAL 1987 LTD., Jerusalem (IL)

(72) Inventors: Nir Weiss, Rehovot (IL); Mark Golberg, Ashdod (IL); Neri Naveh, Kiryat Ono (IL); Oded Levin, Kiryat Ono (IL)

(21) Appl. No.: 14/616,866

(22) Filed: Feb. 9, 2015

(51) Int. Cl. A61B 5/083; A61B 5/00; A61B 5/097

(52) U.S. Cl. CPC A61B 5/0836; A61B 5/097; A61B 5/002

(57) ABSTRACT

The present disclosure provides portable capnography systems having multiple operational modes, with varying power consumption. Alternation between the operational modes may be conducted to reduce the overall power consumption of capnography systems. The power reduction may be advantageous in enabling capnography systems to be powered by mobile power sources.

### TECHNICAL FIELD

The present disclosure generally relates to the field of respiratory monitoring.

### BACKGROUND

Capnography, the monitoring of respiratory CO₂ levels, enables objective evaluation of the ventilatory/respiratory status of a patient, and indirectly enables evaluation of the circulatory and metabolic status. Capnography is used in operation rooms, intensive care units, recovery (post-anesthesia care units), Sedation, Gastroscopy, resuscitation (CPR), emergency departments and others.

Capnography generally requires the operation of advanced complex sensory, pumps and analyzing units, in addition to tubes and electricity-conducting wires to provide the capnography system with the required electrical power for its operation. The existence of the tubes and electricity-conducting wires imposes some limitations, compromising the ease of use of capnography systems, restricting the mobility of the patient, and restraining accessibility to their surrounding environment, including the ability of medical professionals to access the patient for treatment.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiment, there are provided herein devices, systems and methods for increasing mobility and accessibility related to current capnography systems. According to some embodiment, there are provided devices, systems and methods for reducing the power consumption of a portable capnography system. Such a reduction in power consumption may enable an operation of the portable capnography system using a mobile power source to provide power to the portable capnography system, thus reducing or eliminating the need for having the system connected to a power plug.

According to some embodiments, power reduction can be achieved by utilizing portable capnography system components (for example, portable sensors, portable pumps, portable analyzers, and other portable components) that have multiple operational modes, which vary in their power consumption, and introducing a portable controller to select and alternate between the operational modes. According to some embodiments, the portable controller may be configured to select operational modes characterized with higher power consumption when needed, and to select operational modes characterized with less power consumption otherwise, potentially resulting in lower overall power consumption than in traditional operation.

In general, some components of a portable capnography system may consume more power when operated using their full operational capacity or a major part of it. Such an operation may be needed when the capnography system is actively monitoring respiratory signals and may be referred to as “high power mode”. In other time intervals, such a high power mode may be unnecessary. Therefore, some components of the portable capnography system may be operated using less than their full operational capacity or a major part of it, and as a result consume less power. Such operational mode may be referred to as “low power mode(s)”. Thus, the systems and methods disclosed herein, according to some embodiments, advantageously provide a control over the power consumption resulting in energy saving.

The methods and systems disclosed herein, according to some embodiments, may advantageously prolong service life of some components in the portable capnography system compared to currently known Capnography systems. This may optionally be achieved due to the operation of the one or more components of the portable Capnography system in multiple activity levels instead of a regular constant high-level activity.

According to some embodiments, the portable capnography system may include a patient interface unit, a portable tubing system for collecting samples of air from exhaled breath of a subject (patient), a portable carbon dioxide sensor, a portable pump for delivering breath samples from the patient to the portable carbon dioxide sensor, a portable analyses unit for analyzing signals obtained from the portable carbon dioxide sensor, and a portable control circuitry. According to some embodiments, at least one of the portable carbon dioxide sensor, portable pump and portable analysis unit associated with the portable capnography system has at least a first and a second operational modes, and the control circuitry alternates between the operational modes.

According to some embodiments, the portable capnography system may be configured to operate using portable batteries as a power source. Alternatively or additionally, other mobile power sources may be utilized, including ultra-capacitors, super-capacitors, semi-conductor capacitors, carbon fiber batteries and other electro-chemical and electrical mobile power sources.

According to some embodiments, the portable capnography system may further include a portable monitor for displaying information related to the carbon dioxide measurements, the pump, the analysis unit and any information regarding the capnography and the state of the patient.

According to some embodiments, the terms “patient” and “subject” may be interchangeably used.

According to some embodiments, a portable capnography system, includes a portable patient interface unit configured to receive samples of air from an exhaled breath of a subject, a portable carbon dioxide (CO₂) sensor, a portable pump configured to deliver breath samples from the patient interface unit to the portable CO₂ sensor, and a portable control circuitry configured to analyze signals obtained from the portable CO₂ sensor. In which wherein at least one of the portable CO2 sensor, portable pump and portable control circuitry have at least a first and a second operational modes, and the portable control circuitry is configured to alternate between the at least first and second operational modes of at least one of the portable CO₂ sensor, the portable pump and the portable control circuitry.

According to some embodiments, the CO₂ sensor, pump and/or control circuitry consume more energy in the second operational mode thereof than in

According to some embodiments, the first operational mode is an OFF mode.

According to some embodiments, the first operational mode is a STAND BY mode.

According to some embodiments, the second operational mode of the CO₂ sensor, pump and/or control circuitry is activated for a periodic periods of time, for example, once in every minute, 5 minutes, 10 minutes, 15 minutes or every 5-15 minutes.

According to some embodiments, alteration between the first and second operational mode of the CO₂ sensor, pump and/or control circuitry is determined based on a respiratory status of the subject.

According to some embodiments, the portable control circuitry contains a signal sampler having a first and a second sampling rate, the first sampling rate being associated with the first operational mode and the second sampling being associated with the second operational mode.

According to some embodiments, the portable capnography system is configured to receive energy from a mobile energy source.

According to some embodiments, the mobile energy source is rechargeable.

According to some embodiments, the mobile energy source is a portable battery and/or a capacitor.

According to some embodiments, the portable capnography system includes a portable monitor configured to display information related to the portable CO₂ sensor, the portable pump and/or the portable control circuitry.

According to some embodiments, the portable capnography system includes a housing package configured to be attached on a limb of a subject.

According to some embodiments, the portable capnography system includes an alarm configured to alert the subject when an attention of the subject is required.

According to some embodiments, the portable capnography system is configured to receive energy from a mobile energy source, and the attention of the subject is required when a mobile energy source needs recharging.

According to some embodiments, a method is provided for reducing power consumption in a portable capnography system, the method includes alternating between at least a first and a second operational modes of the portable capnography system.

According to some embodiments, a method is provided in which respiratory phases of a subject affect the alternation between the at least first and second operational modes of the portable capnography system.

According to some embodiments, a method is provided in which a respiratory rate a subject affects alternation between the at least first and second operational modes of the portable capnography system.

According to some embodiments, a method is provided in which a the first operational mode is selected as a result of a respiratory rate below a first threshold rate and the second operational mode is selected as a result of a respiratory rate above a second threshold.

According to some embodiments, a method is provided in which the first threshold rate and the second threshold rate are equal.

According to some embodiments, the portable capnography system may be portable and thus facilitate mobility of a patient, even during monitoring.

According to some embodiments, the controller may be configured to alternate between the various operational modes in correlation with different phases of a respiratory cycle of a patient.

According to other possible embodiments, the controller may be configured to select an operational mode in which the portable capnography system operates depending on the respiratory status of a patient.

According to some embodiments, samples are analyzed, and then a decision is made as to whether to operate the portable capnography system in the first operational mode or the second operational mode.

The disclosure is not limited to portable capnography systems having discrete operational modes. The operation of some components may be controlled in a continuous or semi-continuous manner, and as a result, the controller may select a level of operation from continuous or semi-continuous options of power usage modes.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a traditional environment of a patient under capnography monitoring;

FIG. 2 schematically illustrates a wireless portable capnography monitoring, according to some embodiments;

FIG. 3 schematically illustrates a mobile wireless portable capnography system, according to some embodiments;

FIG. 4 schematically illustrates a wireless portable capnography system, according to some embodiments;

FIG. 5 schematically illustrates a wireless portable capnography system, according to some embodiments;

FIG. 6 schematically illustrates power consumption of a wireless portable capnography system at certain operational modes, according to some embodiments;

FIG. 7 schematically illustrates power consumption of a wireless portable capnography system over time, at certain operational modes, according to some embodiments;

FIG. 8 schematically illustrates a method for alternating power modes, according to some embodiments; and

FIG. 9 schematically illustrates a method for alternating power modes, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

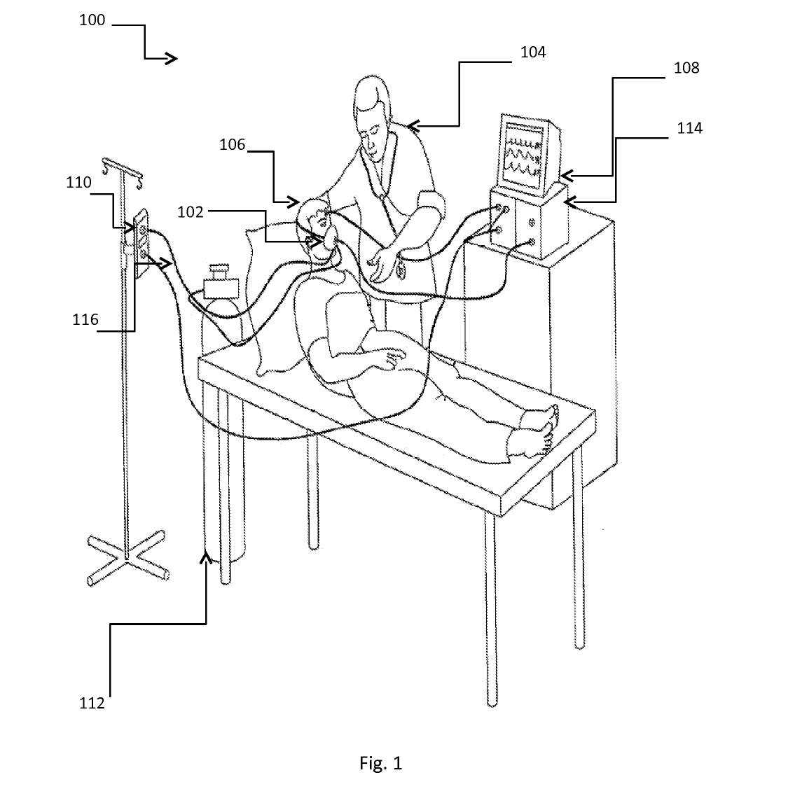

Reference is now made to FIG. 1, which schematically illustrates a traditional environment of a patient under capnography monitoring 100. The patient 106 is connected to a portable capnography system 114 via a patient-interface-unit 102 which delivers exhaled air samples through a tubing system 116 to a sensor 110. Sensor 110 is connected to a control circuitry 108 for analyzing the data delivered by sensor 110. Patient 106 is generally also connected to a pump 112 via additional tubes. A health care provider 104 attempting to provide diagnosis, monitoring or treatment to the patient faces a complex environment resulting partially from the existence of the wires and tubes.

A patient environment having fewer cables, tubes and other mobility limiting objects is desirable. This may result in a more convenient atmosphere for a patient, already under several potential inconveniences resulting from recovery or treatment operations and procedures.

Furthermore, from the standpoint of the health care provider (for example, physician, nurse, paramedic, and others), more accessibility to the patient is desirable. This may be either for providing more convenience to the health care provider, or in more acute situations it may even have an effect on the treatment itself by enabling more accessibility for provider to the patient especially in the case where several health care providers are required or desired to be situated in the surrounding environment of the patient.

Advantageously, and according to some embodiments, the portable capnography system may be made mobile and thus allow Capnography monitoring while the patient is engaged in every day activity, or other activities that might be enabled by such mobility of the system.

In addition to the mobility, the portable capnography system, according to some embodiments, may be wirelessly connected to other device(s). An example for such a wireless connection may be between the portable capnography system and a wireless (smart) phone of the patient. The wireless connection may enable remote controlling of the portable capnography system, and according to some embodiments wireless connection to a computer network is introduced, and advantageously may enable a health care provider to receive information related to the portable capnography over the network.

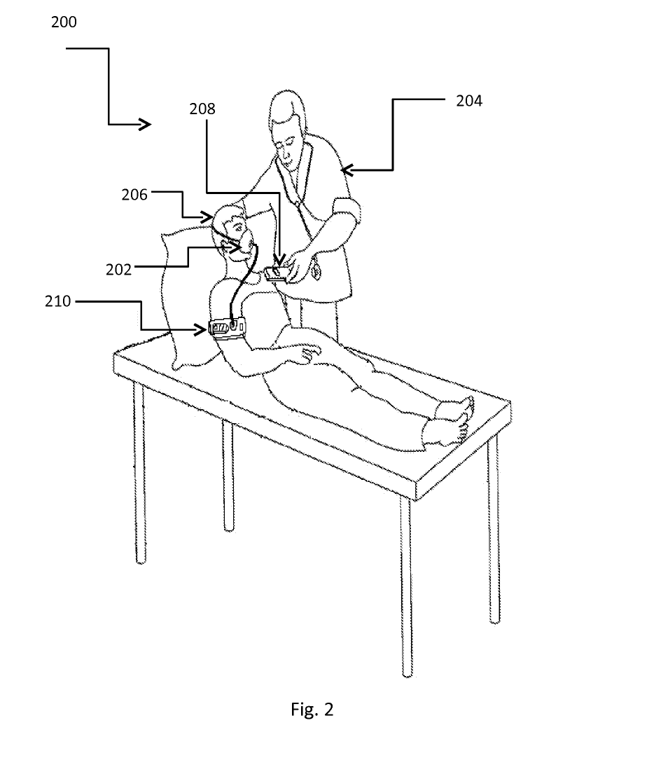

Reference is now made to FIG. 2, which schematically illustrates a wireless capnography monitoring, according to some embodiments. A patient interface unit 202 is attached to the patient 206, being treated, diagnosed or monitored. Patient interface unit 202 is connected to a mobile capnography system 210 comprising a pump, a CO₂ sensor and a monitor, capnography system 210 is wirelessly connected to a wireless handheld device 208. A medical care provider 204 can operate mobile capnography system 210 using handheld device 208, making essential use of its functions while still maintaining a convenient environment 200 for conducting medical procedures that might be needed.

Advantageously, the mobility and the wireless capabilities of the capnography, according to some embodiments, contribute to the ease of use thereof and may make it easier for utilizing the capnography system without an assistance from a health care provider.

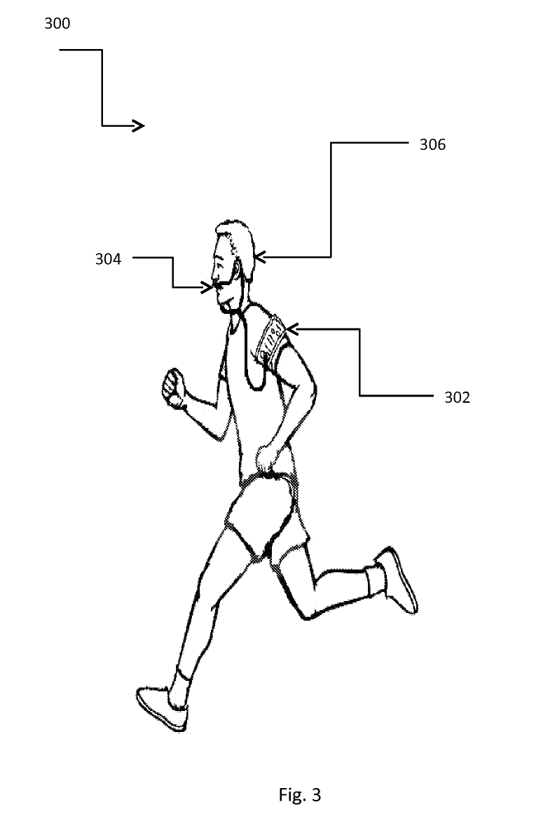

Reference is now made to FIG. 3, which schematically illustrates a mobile wireless capnography system, according to some embodiments, in which a subject 306 is connected to a mobile capnography system 302 by a cannula 304. Subject 306 is able to practice an activity of choice 300 while having his/her CO₂ levels monitored by capnography system 302.

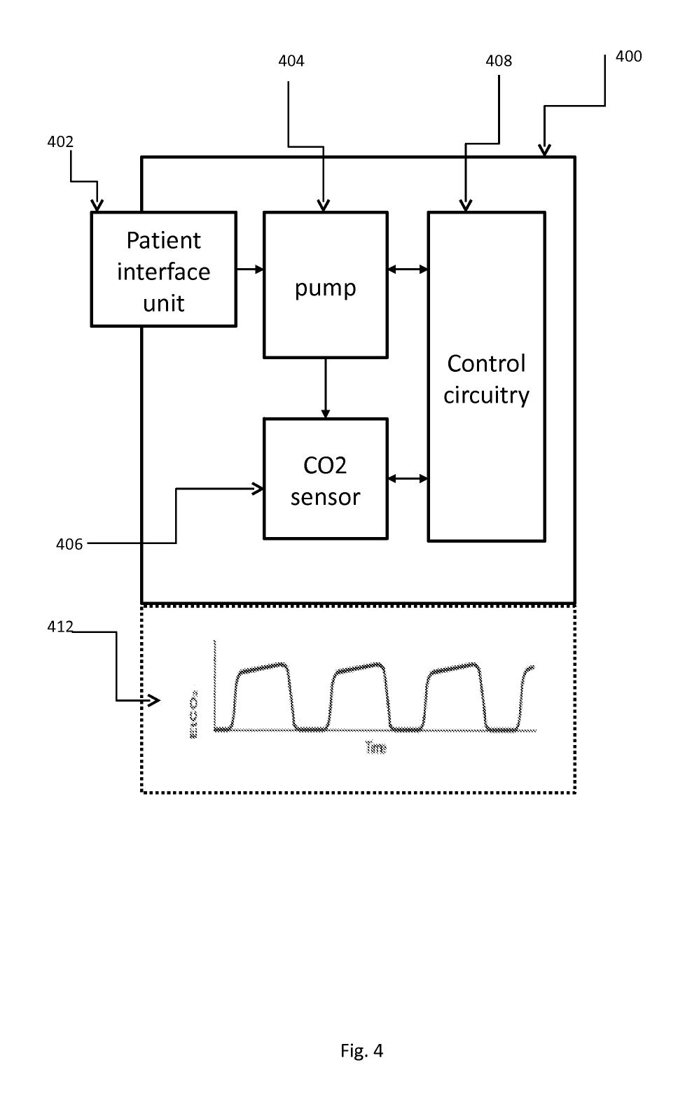

Reference is now made to FIG. 4, which schematically illustrates a wireless capnography system, according to some embodiments a capnography system 400 including a patient interface unit 402, which is configured to receive samples of air from an exhaled breath of a patient. Patient interface unit 402 may be configured to receive samples of exhaled breath from the nose of the patient, the mouth or both. The patient interface unit may comprise an oral/nasal cannula, a face mask, an airway adaptor, a bite block and/or any interface that may collect samples of air from an exhaled breath of a subject.

Capnography system 400 further includes a pump 404, to deliver samples of exhaled air from patient interface 402 unit to a CO₂ sensor 406. Pump 404 may be of any form that enables such delivery of air samples, for example a peristaltic pump, vacuum pump or any other pump, may be utilized to function as a pump. CO₂ sensor 406 may be an infer-red CO₂ sensor, a chemical CO₂ sensor or any other sensor for detecting CO₂ levels in samples.

A control circuitry 408 is also utilized in capnography system 400, according to some embodiments. Control circuitry 408 may include a processor or an analog circuitry or a digital circuitry or any combination thereof, and may further comprise memory elements. The memory elements may contain or be configured to contain software commands or programs to be run by the processor. The memory elements or other memory elements may contain or be configured to contain data relating to the operation of capnography system 400, logs, waveforms and any other information relating to the operation of capnography system 400 or any of its components (for example, data from CO₂ sensor 406, data from pump 404, and others). The use of the term software should not be limiting, as any alternative such as firmware or hardware logic commands may be utilized.

Control circuitry 408, the software and any related data, may be used for the purposes of analyzing the data from CO₂ sensor 406, controlling any component of capnography system 400 or communication purposes that may be needed with other devices. Such communication may be done through wireless or wired means, such as Bluetooth, WIFI, NFC (Near-Field-Communication), Radio or any other wireless or wired communication means.

The communication may be utilized to deliver information, alerts, logs, and other data and/or to receive configuration commands, operational commands and other information that may be required such as software or firmware updated or user information.

According to some embodiments, capnography system 400 may further comprise a monitor 412 for displaying data such as waveform and operational data or any other information that might be relevant to the patient or health care provider. Monitor 412 may provide means for controlling capnography system 400 or monitor 412 itself by using touch-screen technology or any other equivalent technology. Other control means such as switches, buttons knobs and others may be used alternatively or complementarily.

According to some embodiments, in order for the capnography system to consume less energy, this disclosure describes the use of various operational modes of the capnography system or components thereof. An operational mode is a term used to describe a mode in which the capnography system is operated or modes in which components of the capnography system are operated. Each operational mode is correlated to an energy consumption and behavior of the system or components thereof. According to some embodiments, each operational mode is characterized with a different energy consumption and behavior. Alternation between the different operational modes may be done in order to reduce the overall power and energy consumption of the capnography system throughout the monitoring operation.

According to some embodiments, the CO₂ sensor may be operated in at least two different operational modes, a first operational mode and a second operational mode. Optionally, the CO₂ sensor consumes less energy when utilized in the first operational mode than when utilized in the second operational mode. As an example for how this may be achieved according to some embodiments, when the CO₂ sensor is an infrared sensor: operated using the second operational mode, the infrared source of the sensor is configured to emit light with more luminous flux/intensity than when operated using the first operational mode.

According to some embodiments, the pump may be operated in at least two different operational modes, a first operational mode and a second operational mode. Optionally, the pump consumes less energy when utilized in the first operational mode than when utilized in the second operational mode. As an example for how this may be achieved, according to some embodiments, when the operation of the pump is characterized by different air pumping rate and the pumping rate of the pump being operated using the second operational mode is higher than then operated using the first operational mode.

According to some embodiments, the control circuitry, such as control circuitry 408, may be operated using at least two different operational modes, a first operational mode and a second operational mode. Optionally, the control circuitry consumes less energy when utilizing the first operational mode than when utilizing the second operational mode.

According to some embodiments, the control circuitry may include a signal sampler. The sampler signal may be set to sample signals obtained from the CO₂ sensor at a sampling rate. According to some embodiments, a sampling rate is the rate on which the signal sampler samples the data and it is generally quantified in units of samples-per-minute or samples-per-second. Sampling the signal may be needed for example if an analysis of the data is conducted digitally, then the possibly analog signal obtained from the CO₂ sensor is converted to a digital value that is updated every certain period of time according to the sampling rate. The digital data is then delivered to an analysis logic or software running on the control circuitry or parts thereof. Optionally, when using the first operational mode, the sampling rate is lower than when using the second operational mode. Alternatively and perhaps additionally, the processing speed of the analysis logic is lower when using the first operational mode than when using the second operational mode.

Operational modes may relate to modes of operation for a single component, a plurality of components or the operation of the capnography system as a whole. An operational mode of the capnography system may comprise a combination of operational modes or the various components thereof.

According to some embodiments, an ‘active’ and ‘idle’ operational modes are introduced, wherein the ‘active’ mode is characterized with high power consumption and enhanced performance while the ‘idle’ mode is characterized with low power consumption and degraded performance. Advantageously, the control circuitry may choose to utilize the ‘active’ operational mode when a need for enhanced performance is detected and utilize the ‘idle’ mode otherwise. According to some embodiments, the control circuitry may alternate the usage of the ‘active’ mode and the ‘idle’ mode periodically, according to predetermined time intervals, or adaptively adjust the time interval according to meet a temporal need for accuracy or power saving of the capnography system. According to some embodiments, the time intervals are configurable. According to some embodiments, a user may control the time intervals.

According to come embodiments, a user of the capnography system may control the power consumption thereof by configuring the performance of the ‘active’ mode and/or the performance of the ‘idle’ mode.

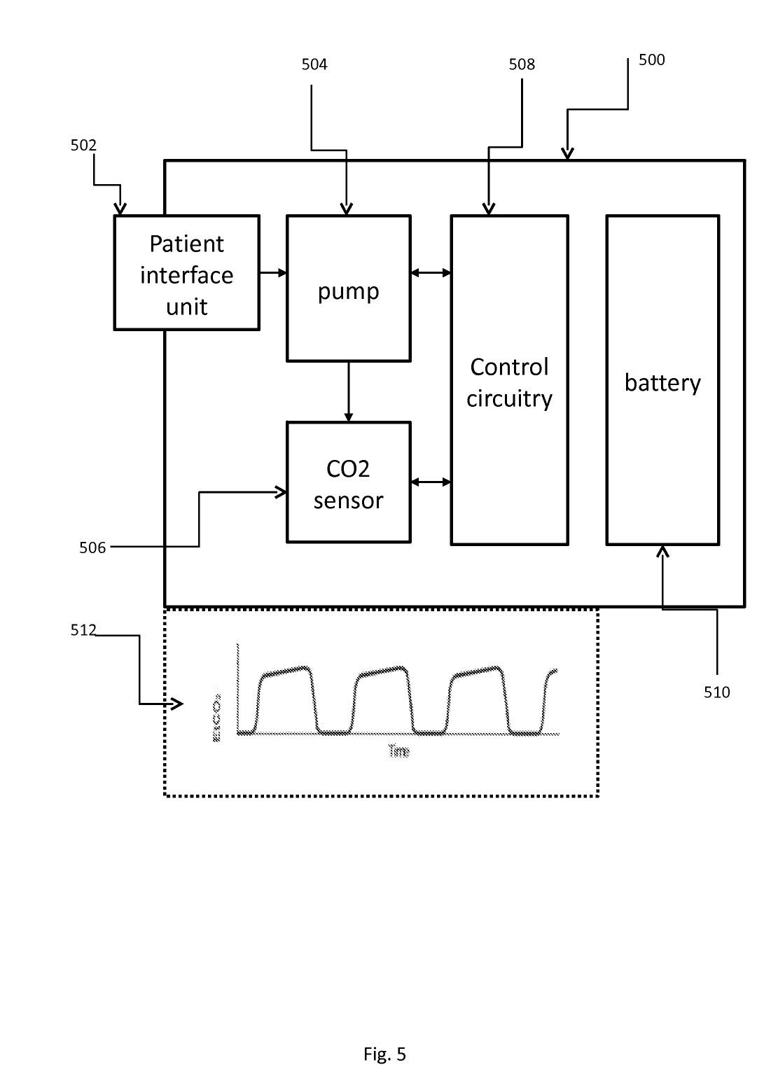

Reference is now made to FIG. 5, which schematically illustrates a capnography system 500 comprising a battery 510. According to possible embodiments, capnography system 500 is configured to receive power for its operation from a mobile power source. The power source is capable of storing power sufficiently for an operation of capnography system 500. Such power source may be battery 510 (for example, Lithium-Ion battery or any other battery), capacitors (for example, supercapacitors, ultracapacitors, carbon-fiber structures, for example,) or any other mobile power source that may sufficiently provide power for the operation of capnography system 500. Battery 510 may be rechargeable, replaceable, disposable or any combination thereof. Capnography system 500 further comprises a patient interface unit 502, a pump 504, a CO₂ sensor 506, a control circuitry 508, and optionally a monitor 512. Patient interface unit 502, pump 504, CO₂ sensor 506, control circuitry 508, and monitor 512 may have a form and/or a function essentially as mentioned in the description of corresponding elements in FIG. 4.

According to some embodiments, the capnography system may be mobile. The mobility may be enabled partially by providing the capnography system with a mobile power source, such as battery 510, and eliminating the requirement for constant connection to an electric-power outlet. According to some embodiments, the capnography system may be held by the patient or even attached to a limb of a patient, for example by using a capnography housing that can be attached to a patient limb by a stripe or a clips or any other means for attaching the capnography to the patient or the patient's clothing or other possible carrying methods.

The mobility may advantageously, according to some embodiments, enable a wider range of usage of capnography systems. For instance, capnography operation may be enabled for usage of the patient while in physical activity such as walking or cycling or other activities that might require mobility of the patient. Other enabled possible uses may be the usage of the capnography system at the residence of the patient for capnography monitoring across long periods of time, or periodic capnography monitoring without the need for the patient to be available at a clinic or hospital for performing each capnography monitoring.

The power reduction may result in other advantages not necessarily mentioned in this disclosure, but may be apparent to a person skilled in the field of capnography. One such resulting advantage may be the ability to operate the capnography system for longer periods using a reserve power supply, in case of an electric power down of the regular electricity supply.

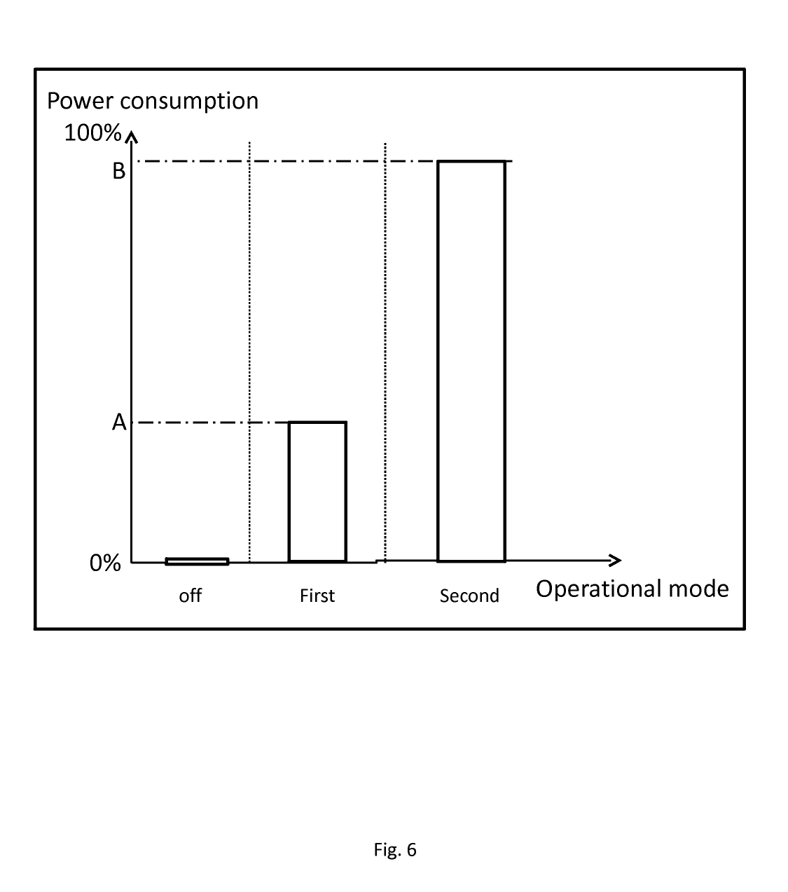

Reference is now made to FIG. 6, which schematically illustrates power consumption of a capnography system at certain operational modes. When the capnography system is operated using the first operational mode, the power consumption of the system is A, while when the capnography system is operated using the second operational mode, the power consumption of the system is B. As shown in the graph, A is a lower power consumption level than B, meaning that the capnography system consumes less power when operated in the first operational mode than when operated in the second operational mode. When the capnography system is ‘off’ the power consumption thereof is fairly low or null. The power consumption difference between A and B is advantageously used to save power during the operation of the capnography system.

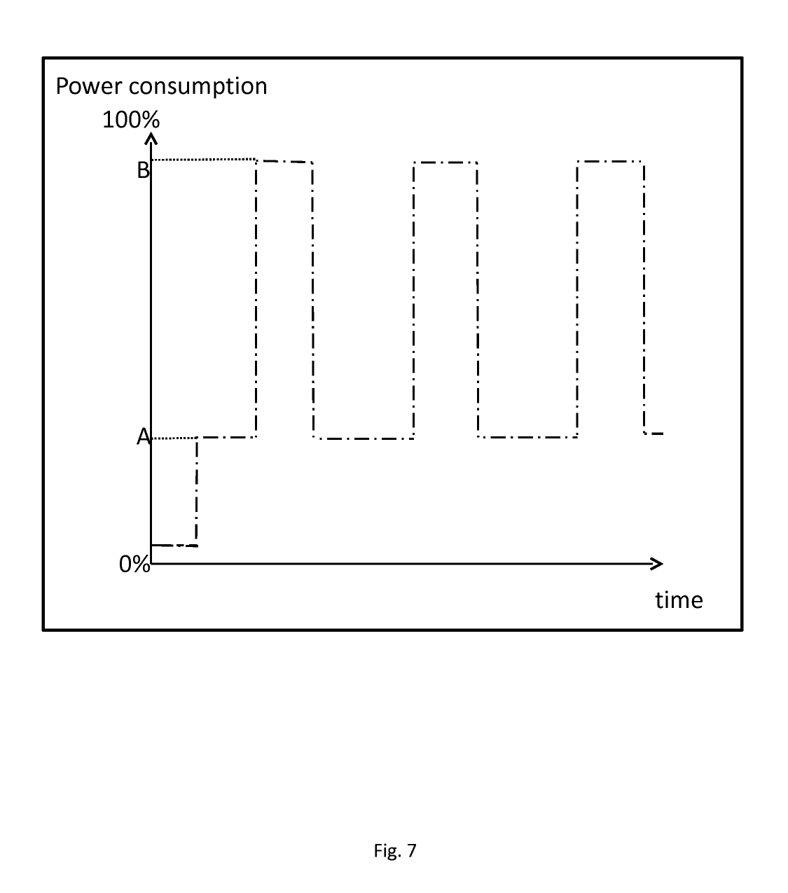

Reference is now made to FIG. 7, which schematically illustrates power consumption of a capnography system across time, utilizing different operational modes. After the capnography system is turned on, a control circuitry conducts a periodic alternation between a first operational mode and a second operational mode, resulting in a power consumption reduction compared to a capnography system that operates using a single high-power consuming operational mode.

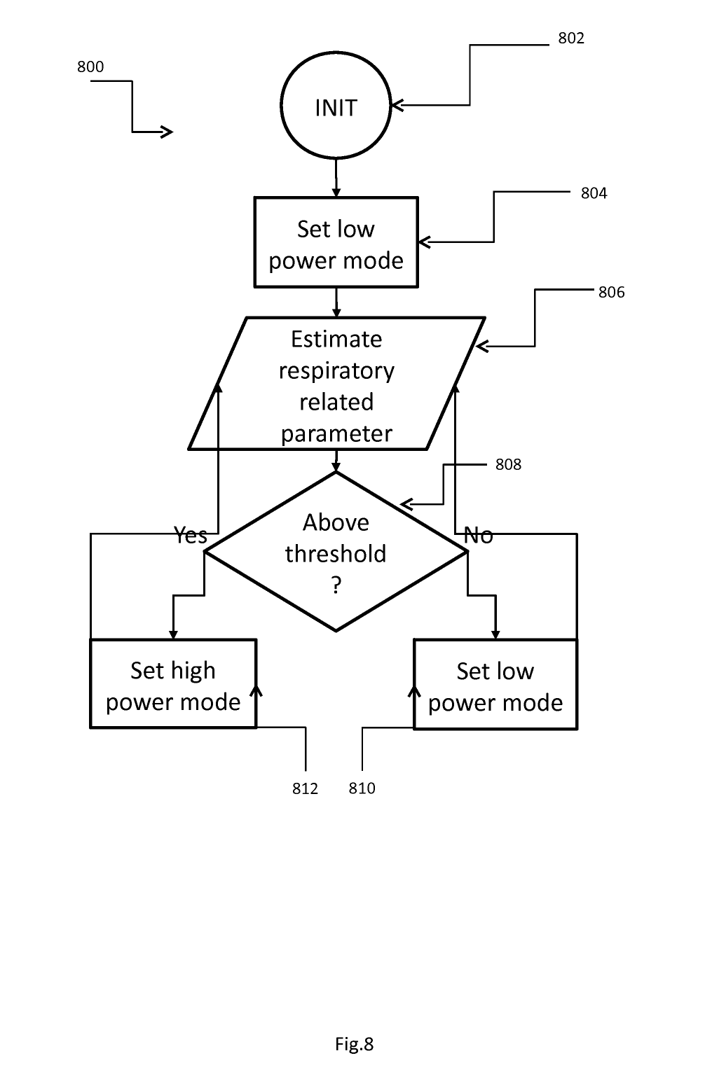

Reference is now made to FIG. 8, which schematically illustrates a method for alternating power modes based on respiratory rate estimation. A flow-chart 800 is introduced according to some embodiments, beginning in an initial state 802; setting a low power operational mode 804; detecting or estimating respiratory related parameter(s) 806 obtained by a CO₂ sensor, a respiratory activity index may be a respiratory rate of a patient; deciding whether a high power operational mode is needed by comparing the estimated respiratory parameter(s) to a pre-defined or configurable threshold 808; either set a high power operational mode 812 or set a low power operational mode 810 according to the decision.

The analysis and decision criteria may vary across different uses of the capnography system, the choice of the components, configuration and other conditions. The patient related parameter(s) estimation may be used for deciding whether a high-power mode or low power mode are needed.

The patient related parameter(s) may include, for example any parameter indicative of the patient's status/condition.

The patient related parameter(s) may include, for example (but not limited to): breath related parameters, such as, for example, respiratory rate, CO₂ related parameters, and the like; O₂ related parameters, such as, for example, SpO2, O₂ saturation, and the like; heart related parameters, such as, for example, heart rate, ECG, blood pressure, and the like; neurological parameters, such as, for example, EEG; spirometry related parameters, such as, for example, FEV1, FVC, and the like; and the like.

The patient related parameter(s) may include, for example (but not limited to) an index (patient related index) indicative of the patient's status/condition.

According to some embodiments, the term “index” may refer to a “condition-index-value”, “respiratory index”, “pulmonary index value”, “health index” and/or “index value”, which may interchangeably be used.

As referred to herein, the term “pulmonary” includes relating to, affecting, or occurring in the lungs.

As referred to herein, the term “respiratory” relates to the system that consists of or includes the airways, the lungs, and the respiratory muscles that mediate the movement of air into and out of the body.

According to some embodiments, the term “pulmonary index value” may refer to a pulmonary index and/or a respiratory index. The term “pulmonary index value” may further relate to a respiratory and cardiac index and/or to a pulmonary and cardiac index.

A patient related-index value may be determined/calculated/computed based upon various parameters of a patient that may be sensed/measured by appropriate sensors. The various parameters, according to which the condition index value is determined, may each have different units and occasionally, different units may be used for the same parameter. Moreover, the absolute values of the parameters may not always be intuitive for understanding/interpretation and neither are they linearly proportional to severity of the condition. In addition, some parameters may have different meanings as to the condition of the patient when increasing and/or when decreasing, that is, for some parameters, decrease in the value indicates improvement while with other parameters, decrease in value may indicate deterioration of the patient condition. This further demonstrate the importance of a condition index value, which integrates various parameters that may be measured in different units and may have different meanings into one comprehensible index value, which may be indicative of the absolute patient condition.

According to some embodiments, the patient related-index-value may be a unit-less value in any predetermined range, such as, for example, in the range of 1 to 100. For example, condition-index-value may in the range of 1 to 10, wherein 10 indicates the best condition, and 1 indicates the worst condition. Within the range of 1 to 10, sub ranges (subdivisions) may be assigned. For example, a sub-range from 8 to 10 may be indicative of a stable, normal condition, where no intervention is needed. A sub-range of 6-7 may be indicative to the health care provider that more attention is needed patient re-evaluation is recommended. A sub range of below 5 may indicate to the health care provider that intervention is needed and patient re-evaluation may be necessary. In addition, the various sub-ranges of the condition-index-value may be assigned different graphical signs, when displayed to the health care provider. The different graphical signs may include, for example, different colors, different units, different letters, and the like. For example, for condition-index-value in the sub-range of 8 to 10, the value may be colored green, for condition-index-value in the sub-range of 5 to 7, the value may be colored yellow, and for condition-index-value in the sub-range of below 5, the value may be colored red. In addition, various other visual indicators may also be used to indicate changes that may be correlated with known physical conditions, such as, for example, up and down arrows that may indicate, for example, an increase or decrease, respectively, in one or more measured parameters.

An example for an optional analysis and decision method may be as follows: an analysis is conducted to determine or estimate the respiratory rate of the patient; if the respiratory rate is below a certain threshold, a first operational mode is selected; and if the respiratory rate is above a certain threshold, a second operational mode is selected. The thresholds may be set at different values, or alternatively have the same value. Setting the thresholds to different values may result in a hysteresis behavior, which may be a desirable behavior to eliminate unnecessary alternation resulting from minute changes of the respiratory rate near a certain level.

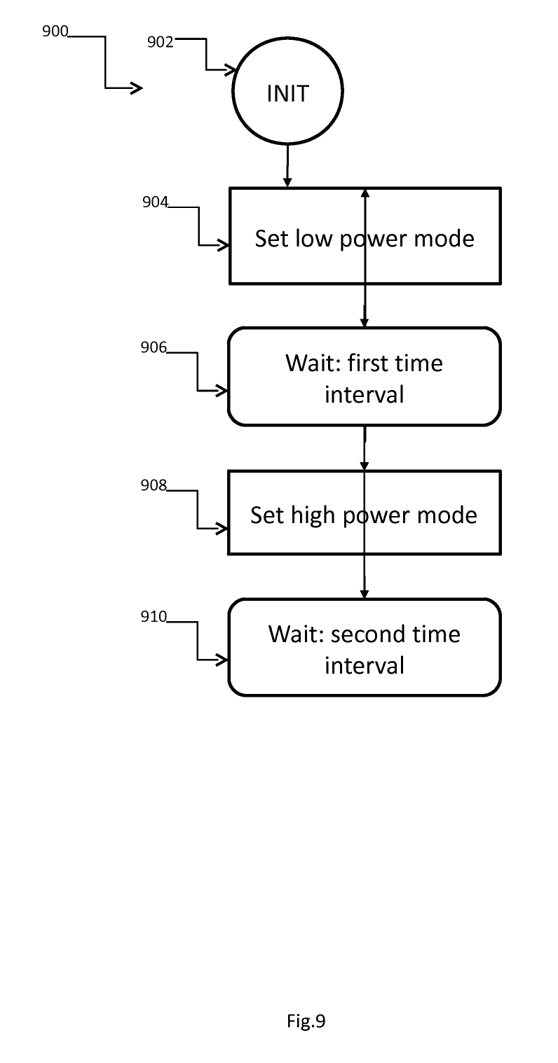

Reference is now made to FIG. 9, which schematically illustrates a method for periodically alternating power in a capnography system modes using predetermined time periods. A flow-chart 900 is introduced according to some embodiments, beginning in an initial state 902; setting a low power operational mode 904; waiting for a “first time interval” period of time 906; setting a high power operational mode 908; waiting a “second time interval” period of time 910; getting back to setting low power operational mode 904; and so forth until interrupted externally or internally.

The “first time interval” is the period of time during which the low power operational mode is selected each cycle, and the “second time interval” is the period of time during which the high power operational mode is selected each cycle.

The result is a periodic alternation pattern between a high power operational 908 mode and a low power operational mode 904, each being selected for a predetermined period of time. “first time interval” period 910 and “second time interval” period 906 may be either different values or alternatively equal values. More operational modes may be applicable and therefore more wait times may apply.

The figures and description may relate to the capnography system as a whole or to any component or combination of components thereof.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A portable capnography system, comprising:
a patient interface unit, configured to receive samples of air from an exhaled breath of a subject;
a portable carbon dioxide (CO₂) sensor;
a portable pump, configured to deliver breath samples from said patient interface unit to said portable CO₂ sensor; and
a portable control circuitry configured to analyze signals obtained from said CO₂ sensor,
wherein at least one of said portable CO₂ sensor, portable pump and portable control circuitry have at least a first and a second operational modes, and said control circuitry is configured to alternate between said at least first and second operational modes of at least one of said portable CO₂ sensor, said portable pump and said portable control circuitry.

2. The portable capnography system of claim 1, wherein said portable CO₂ sensor, portable pump and/or portable control circuitry consume more energy in said second operational mode thereof than in said first operational mode thereof.

3. The portable capnography system of claim 1, wherein said first operational mode is an OFF mode.

4. The portable capnography system of claim 1, wherein said first operational mode is a STAND BY mode.

5. The portable capnography system of claim 1, wherein said second operational mode of said portable CO₂ sensor, portable pump and/or portable control circuitry is activated for a periodic periods of time.

6. The portable capnography system of claim 1, wherein alteration between said first and second operational mode of said portable CO₂ sensor, portable pump and/or portable control circuitry is determined based on a respiratory status of said subject.

7. The portable capnography system of claim 1, wherein said control circuitry comprises a signal sampler having a first and a second sampling rate, said first sampling rate being associated with said first operational mode and said second sampling being associated with said second operational mode.

8. The portable capnography system of claim 1, configured to receive energy from a mobile energy source.

9. The portable capnography system of claim 8, wherein said mobile energy source is rechargeable.

10. The portable capnography system of claim 8, wherein said mobile energy source comprises a portable battery and/or a portable capacitor.

11. The portable capnography system of claim 1, further comprising a monitor configured to display information related to said portable CO₂ sensor, said portable pump and/or said portable control circuitry.

12. The portable capnography system of claim 1, wherein said capnography system is portable, such that it permits mobility of said subject while utilizing said capnography system.

13. The portable capnography system of claim 1, further comprising a housing package configured to be attached on a limb of a subject.

14. The portable capnography system of claim 1, further comprising an alarm configured to alert said subject when an attention of said subject is required.

15. The portable capnography system of claim 14, configured to receive energy from a mobile energy source, and said attention of said subject is required when a mobile energy source needs recharging.

16. A method for reducing power consumption of a portable capnography system, the method comprises:
alternating between at least a first and a second operational modes of said portable capnography system.

17. The method of claim 16, wherein respiratory phases of a subject affect said alternation between said at least first and second operational modes of said portable capnography system.

18. The method of claim 16, wherein a respiratory rate a subject affects alternation between said at least first and second operational modes of said portable capnography system.

19. The method of claim 18, wherein a said first operational mode is selected as a result of a respiratory rate below a first threshold rate and said second operational mode is selected as a result of a respiratory rate above a second threshold.

20. The method of claim 19, wherein said first threshold rate and said second threshold rate are equal.
